(19) United States
(12) Patent Application Publication
(10) Pub. No.: US 2026/0199196 A1
(43) Pub. Date: Jul. 16, 2026

(54) COSMETIC PRODUCT FOR SPRAY APPLICATION

(71) Applicant: HENKEL AG & CO. KGAA, Düsseldorf (DE)

(72) Inventors: Tim BETHGE, Hamburg (DE); Rolf BAYERSDOERFER, Hamburg (DE); Diane METTEN, Hamburg (DE); Silke HANSEN, Pinneberg (DE)

(21) Appl. No.: 19/128,948

(22) PCT Filed: Jul. 10, 2023
(86) PCT No.: PCT/EP2023/069090

§ 371 Date: May 9, 2025

(30) Foreign Application Priority Data
Nov. 15, 2022 (DE) 10 2022 212 076.0

(51) Int. Cl. A45D 34/04

(52) U.S. Cl. CPC A61K 8/046; A45D 2200/056

(57) ABSTRACT

Cosmetic products for spray application are described, which have advantages in terms of variability to spray different preparations with one device, resource consumption, even application, and effectiveness alongside small quantities being used.

The present invention relates to a cosmetic product comprising a spray application device and a cosmetic preparation to be sprayed.

The spray application of cosmetic compositions is a proven state of technical knowledge. Corresponding products are widely commercially available in the cosmetics sector as well as in many other fields of application. The majority of such products are based on a non-reusable pressure container containing one or more propellants and the cosmetic preparation to be sprayed. By operating the valve, the propellant expands out of the pressure container and sprays the cosmetic preparation via suitable nozzles.

Alternative solutions include pump-type spray devices, which can be refilled as needed, thus reducing the amount of packaging used. However, in addition to their potential to improve sustainability, both solutions also have the potential to improve the spray pattern and variability, for example to be able to spray different cosmetic preparations (deodorant, hairspray, etc.).

A novel approach to spraying is disclosed in European patent applications EP 3 229 752 A1, EP 3 229 917 A1 and EP 3 22 991 A1. According to the teaching in these documents, cosmetic compositions are sprayed by means of a device for flash evaporation, which comprises a container that defines a closed interior in which the cosmetic preparation can be accommodated, a valve or a closure element operating in a similar manner in order to close and open the interior of the container, which is at least partially filled with the cosmetic preparation, a heating device in order to heat the cosmetic preparation in the closed interior of the container under increased pressure, as well as to release the heated cosmetic preparation from the interior of the container under reduced pressure into the environment, and a nozzle which allows atomization of the cosmetic preparation discharged from the container.

The object of the present invention was to develop the field of cosmetic products for spray application. Particular emphasis was to be placed on variability (the ability to spray different preparations using one device), resource consumption, the most uniform application possible and high effectiveness together with small application quantities.

It has now been found that devices with special cosmetic preparations whose physical properties are tailored to the device solve this problem.

The present invention relates, in a first embodiment, to a cosmetic product comprising
a) a cosmetic preparation,
b) a device for the finely distributed application of the cosmetic preparation a), comprising
   b1) a storage container for the cosmetic preparation a),
   b2) a pump or a conveying element operating in a similar manner for conveying the cosmetic preparation a) out of the storage container b1);
   b3) a pressure chamber in which the cosmetic preparation a) conveyed out of the storage container b1) can be subjected to a pressure increase, comprising
      b3a) an inlet valve or a closure element operating in a similar manner in order to be able to seal the cosmetic preparation a) conveyed by means of the conveying element b2) from the storage container b1) into the pressure chamber b3) with respect to the environment;
      b3b) an outlet valve or a closure element operating in a similar manner in order to be able to apply the cosmetic preparation a) from the pressure chamber b3) that is under increased pressure in a finely distributed manner,
wherein the cosmetic preparation a) has a degree of thermal conductivity of 0.02 to 2.0 Wm⁻¹K⁻¹ at 25° C.

The present invention also relates to a method for spraying a cosmetic preparation, in which
a) a cosmetic preparation is sprayed by means of
b) a device for the finely distributed application of the cosmetic preparation a), comprising
   b1) a storage container for the cosmetic preparation a),
   b2) a pump or a conveying element operating in a similar manner for conveying the cosmetic preparation a) out of the storage container b1);
   b3) a pressure chamber in which the cosmetic preparation a) conveyed out of the storage container b1) can be subjected to a pressure increase, comprising
      b3a) an inlet valve or a closure element operating in a similar manner in order to be able to seal the cosmetic preparation a) conveyed by means of the conveying element b2) from the storage container b1) into the pressure chamber b3) with respect to the environment;
      b3b) an outlet valve or a closure element operating in a similar manner in order to be able to apply the cosmetic preparation a) from the pressure chamber b3) that is under increased pressure in a finely distributed manner,
wherein the cosmetic preparation a) has a degree of thermal conductivity of 0.02 to 2.0 Wm⁻¹K⁻¹ at 25° C.

The cosmetic product and the method for spraying a preparation will be described in more detail below. The cosmetic preparation a) and the device b) will be disclosed in more detail, and, even if at times only the “cosmetic product” is referred to for linguistic reasons, the method according to the invention is always also being disclosed in more detail. “The product according to the invention comprises” always means “The product according to the invention comprises and is used in the method according to the invention”.

The product according to the invention comprises a cosmetic preparation which has been optimized with regard to its sprayability, whereby the degree of thermal conductivity thereof is 0.02 to 2.0 Wm⁻¹K⁻¹ at 25° C.

Thermal conductivity (sometimes also referred to as the k-value or coefficient of thermal conductivity) is a material property that determines the flow of heat through a material as a result of thermal conduction. Thermal conductivity tells us how well a material conducts heat.

The degree of thermal conductivity of most materials increases slightly with increasing temperature. At a phase transition or state of aggregation transition (e.g., solid↔liquid↔gaseous), the degree of conductivity usually changes significantly and abruptly.

It has been shown that cosmetic compositions can be sprayed particularly evenly so as to be finely distributed by the devices in the product according to the invention if their degree of thermal conductivity is at least 0.02 Wm⁻¹K⁻¹ and does not exceed the value 2.0 Wm⁻¹K⁻¹. Within these limits, narrower value ranges are particularly preferred, and therefore preferred cosmetic products or methods are characterized in that the cosmetic preparation a) has a degree of thermal conductivity of 0.05 to 1.5 Wm⁻¹K⁻¹, preferably of 0.1 to 1.0 Wm⁻¹K⁻¹ and in particular of 0.2 to 0.6 Wm⁻¹K⁻¹ at 25° C.

Within the range of 0.02 to 2.0 Wm⁻¹K⁻¹ and in particular within the narrower ranges mentioned above, the cosmetic compositions can be effectively heated and sprayed and tend neither to clog valves nor to form an uneven spray mist. Lower degrees of thermal conductivity below 0.02 Wm⁻¹K⁻¹ lead to less even spray patterns and high spray losses; at higher degrees of thermal conductivity above 2.0 Wm⁻¹K⁻¹, spray jet widths and homogeneities are unacceptable for a cosmetic application.

The degree of thermal conductivity of a cosmetic composition can be adjusted by adjusting the type and quantity of its constituents. The relationship between thermal conductivity and the content of a substance is not linear, even in binary mixtures (e.g., of water and ethanol); in multi-component mixtures, the degree of thermal conductivity can be adjusted to within the range according to the invention by adding or omitting, increasing or decreasing, the amount of certain constituents.

The product according to the invention can be further optimized with regard to its application properties in that the cosmetic preparation a) has a specific heat capacity at 20° C. of 1.6 to 5.2 Jg⁻¹K⁻¹.

The specific heat capacity of a substance in a given state is the heat added to or removed from a quantity of the substance divided by the corresponding increase or decrease in the temperature and mass of the substance:

$$c = Δ Q / ( m Δ T )$$

For the cosmetic compositions a) in the product according to the invention, the specific heat capacity can be calculated as the quotient of the heat capacity and the mass m of the body. Since heat capacity is an extensive state function, i.e., in the case of bodies composed of parts, it can be calculated as the sum of the heat capacities of its parts; the specific heat capacity of the cosmetic preparation a) can also be calculated if the heat capacities of the individual constituents are known.

It has been found that cosmetic compositions can be sprayed particularly evenly and so as to be finely distributed using the devices in the product according to the invention if their specific heat capacity at 20° C. is at least 1.6 Jg⁻¹K⁻¹ and does not exceed the value 5.2 Jg⁻¹K⁻¹. Within these limits, narrower value ranges are particularly preferred, and therefore preferred cosmetic products or methods are characterized in that the cosmetic preparation a) has a specific heat capacity of 2.1 to 5.0 Jg⁻¹K⁻¹, preferably of 2.6 to 4.6 Jg⁻¹K⁻¹ and in particular of 3.1 to 4.3 Jg⁻¹K⁻¹ at 20° C.

As already mentioned, specific heat capacity is a temperature-dependent variable. It has proven particularly advantageous for the product according to the invention if the specific heat capacity of the cosmetic composition a) lies within a certain range of values at higher temperatures. Here, cosmetic products according to the invention are preferred in which the cosmetic preparation a) has a specific heat capacity of 2.4 to 5.4 Jg⁻¹K⁻¹, preferably of 3.0 to 5.0 Jg⁻¹K⁻¹ and in particular of 3.5 to 4.2 Jg⁻¹K⁻¹ at 50° C.

Within the range of 1.6 to 5.2 Jg⁻¹K⁻¹ and in particular within the narrower ranges mentioned above, the cosmetic compositions can be effectively heated and sprayed and tend neither to clog valves nor to form an uneven spray mist. Lower specific heat capacities below 1.6 Jg⁻¹K⁻¹ lead to less even spray patterns and high spray losses; at higher specific heat capacities above 5.2 Jg⁻¹K⁻¹, spray jet widths and homogeneities are unacceptable for a cosmetic application.

The specific heat capacity of a cosmetic composition can be adjusted by adjusting the type and amount of its constituents; in the case of multi-component mixtures, the specific heat capacity can be adjusted to within the range according to the invention by adding or omitting, increasing or decreasing, the amount of certain constituents.

It has further been found that cosmetic compositions can be sprayed particularly evenly and so as to be finely distributed using the devices in the product according to the invention if their volumetric coefficient of thermal expansion γ (20° C.) is 0.0001 to 0.005 K⁻¹.

The volumetric coefficient of thermal expansion γ is a characteristic value that describes the behavior of a substance or mixture of substances with regard to changes in its dimensions when the temperature changes. The effect responsible for this is thermal expansion. Thermal expansion depends on the material used, and is thus a substance-specific material constant. Since the thermal expansion of many materials does not occur uniformly across all temperature ranges, the volumetric coefficient of thermal expansion itself is also temperature-dependent and is therefore given for a specific reference temperature or a specific temperature range.

With regard to the product according to the invention, the volumetric coefficient of thermal expansion describes γ the change in volume of the cosmetic preparation a) when the temperature increases.

It has been found that cosmetic compositions can be sprayed particularly evenly and so as to be finely distributed using the devices in the product according to the invention if their volumetric coefficient of thermal expansion γ (20° C.) is at least 0.0001 K⁻¹ and does not exceed 0.005 K⁻¹. Within these limits, narrower value ranges are particularly preferred, and therefore preferred cosmetic products or methods are characterized in that the cosmetic preparation a) has a volumetric coefficient of thermal expansion γ (20° C.) from 0.00012 to 0.002 K⁻¹, preferably from 0.00015 to 0.001 K⁻¹, particularly preferably from 0.00017 to 0.00105 K⁻¹ and in particular from 0.0002 to 0.0011 K⁻¹.

The volumetric coefficient of thermal expansion γ is, as already mentioned, a temperature-dependent variable. It has proven particularly advantageous for the product according to the invention if the volumetric coefficient of thermal expansion γ of the cosmetic composition a) is within a certain range of values at higher temperatures. Here, cosmetic products according to the invention are preferred in which the cosmetic preparation a) has a volumetric coefficient of thermal expansion γ (50° C.) of 0.0001 to 0.0024 K⁻¹, preferably from 0.0002 to 0.002 K⁻¹, particularly preferably from 0.0004 to 0.0016 K⁻¹ and in particular from 0.0006 to 0.0012 K⁻¹.

Within the range of 0.0001 to 0.005 K⁻¹ and in particular within the narrower ranges mentioned above, the cosmetic compositions can be effectively heated and sprayed and tend neither to clog valves nor to form an uneven spray mist. Higher volumetric coefficient of thermal expansions above 0.005 K⁻¹ lead to less even spray patterns and high spray losses; at lower volumetric coefficient of thermal expansions below 0.0001 K⁻¹, spray jet widths and homogeneities are unacceptable for a cosmetic application.

Adjusting the volumetric coefficient of thermal expansion γ (20° C.) or the volumetric coefficient of thermal expansion γ (50° C.) of a cosmetic composition can be achieved by adjusting the type and quantity of its constituents; in the case of multi-component mixtures, the volumetric coefficient of thermal expansion γ can be adjusted to within the range according to the invention by adding or omitting, increasing or decreasing, the amount of certain constituents.

The product according to the invention can be further optimized with regard to its application properties by the cosmetic preparation a) having a vapor pressure (according to the Grain-Watson model) of 1200 to 8000 Pa at 20° C.

The vapor pressure describes the equilibrium of a substance between its solid or liquid phase and its gas phase. The vapor pressure is a variable that is heavily dependent on temperature. Temperature dependence is described by the Clausius-Clapeyron equation, in which the vapor pressure is related to the temperature.

$$d ( ln {P}_{v} ) / dT = Δ H / ( {RT}^{2} )$$

The variable ΔH corresponds to the enthalpy of vaporization during transitions from the liquid to the gaseous state. The deviation of the gaseous substance under consideration from the behavior of an ideal gas can be accounted for by means of the compressibility difference between the two phases. This parameter has a value between 1.00 and 0.91 [61].

The model for calculating vapor pressure based on Watson and extended by Grain is applicable to solid and liquid organic chemicals:

$$ln {P}_{v} = Sv / ( Δ {Z}^{*} R ) f ( {T}_{p} , m ) + ln ( corr )$$

$${T}_{p} = T / {T}_{b}$$

$$f = 1 - {( 3 - 2 {T}_{p} )}^{m} / {T}_{p} - 2 {m ( 3 - 2 {T}_{p} )}^{m - 1} ln {T}_{p}$$

For liquids, m=0.19 and the following rule applies for solids: If Tρ is greater than 0.6, m has a value of 0.36; if Tρ is less than 0.5, m=1.19. If Tρ is between 0.5 and 0.6, m is 0.8. Alternatively, m can also be interpolated between 0.38 and 1.19, which is done using a modification by Sage & Sage, where m takes different values depending on Tρ:

$$m = 0.4133 - {0.2575}^{*} T ρ$$

For solids, the same function for f is used as for liquids. Since the Grain-Watson model is also applicable to solids, it does not require fugacity correction by the melting point. Nevertheless, it must be determined whether a substance is solid or liquid at the system temperature.

Using the equations mentioned above (and now also using commercially available computer programs), the vapor pressure of a cosmetic preparation a) can be calculated depending on its composition.

It has been found that cosmetic compositions can be sprayed particularly evenly and so as to be finely distributed by the devices in the product according to the invention if their vapor pressure (according to the Grain-Watson model) is at least 1200 Pa and does not exceed 8000 Pa. Within these limits, narrower value ranges are particularly preferred, and therefore preferred cosmetic products or methods are characterized in that the cosmetic preparation a) has a vapor pressure (according to the Grain-Watson model) of 1600 to 7000 Pa, preferably of 1800 to 6000 Pa and in particular of 2200 to 5600 Pa at 20° C.

Within the range of 1200 to 8000 Pa and in particular within the narrower ranges mentioned above, the cosmetic compositions can be effectively heated and sprayed and tend neither to clog valves nor to form an uneven spray mist. Lower vapor pressures below 1200 Pa lead to less even spray patterns and high spray losses; at higher vapor pressures above 8000 Pa, spray jet widths and homogeneities are unacceptable for a cosmetic application.

The vapor pressure of a cosmetic composition can be adjusted by adjusting the type and amount of its constituents; in the case of multi-component mixtures, the vapor pressure can be adjusted to within the range according to the invention by adding or omitting, increasing or decreasing, the amount of certain constituents.

The product according to the invention can be further optimized with regard to its application properties by adapting the viscosity of the cosmetic preparation a) to the apparatus environment.

Viscosity is a measure of the internal flow resistance (“the thickness”) of a cosmetic preparation a). Viscosity is a temperature-dependent variable which is measured at 20° C. within the context of the present invention. Since the viscosity measurement of the same preparation at the same temperature also shows a certain dependence on the apparatus used, viscosity values within the scope of the present invention are uniformly related to a measurement with a Brookfield DV2T viscometer (and, as mentioned, at 20° C.), whereby the other apparatus parameters (speed, spindle) are adapted to the viscosity to be measured each time.

It has proven advantageous to choose low viscosities for certain applications, such as hairsprays or hair setting products. It has been shown that such compositions can be sprayed particularly evenly and so as to be finely distributed using the devices in the product according to the invention if their viscosity (Brookfield DV2T, spindle 2, 10 rpm) at 20° C. is at least 10 mPas and does not exceed 100 mPas. Within these limits, narrower value ranges are particularly preferred, and therefore preferred cosmetic products or methods are characterized in that the cosmetic preparation a) has a viscosity (Brookfield DV2T, spindle 2, 10 rpm) of 20 to 60 mPas at 20° C.

Within the range of 10 to 100 mPas and in particular within the narrower ranges mentioned above, the cosmetic compositions can be effectively heated and sprayed and tend neither to clog valves nor to form an uneven spray mist.

There are other forms of application (e.g., hair treatments, hair conditioners or hair masks) where higher viscosity values have proven to be advantageous. It has been found that such compositions can be sprayed particularly uniformly and so as to be finely distributed using the devices in the product according to the invention if their viscosity at 20° C. (Brookfield DV2T, spindle 5, 20 rpm) is 500 to 25000 mPas. Within these limits, narrower value ranges are particularly preferred, and therefore preferred cosmetic products or methods are characterized in that the cosmetic preparation a) has a viscosity (Brookfield DV2T, spindle 5, 20 rpm) of 1000 to 20000 mPas, preferably of 1500 to 15000 mPas and in particular of 2000 to 10000 mPas at 20° C.

The viscosity of a cosmetic composition can be adjusted by adjusting the type and amount of its constituents; in the case of multi-component mixtures, the viscosity can be adjusted to within the range according to the invention by adding or omitting, increasing or decreasing, the amount of certain constituents.

The product according to the invention can be further optimized with regard to its application properties in that the cosmetic preparation a) contains at least 5 wt. % of a first solvent LM1 which is liquid at 20° C. and at least 5 wt. % of a second solvent LM2 which is liquid at 20° C., wherein the difference between the boiling points of solvents LM1 and LM2 at 1013.25 mbar is in the range from 5 to 50° C.

The product according to the invention therefore contains a mixture of at least two solvents, with the cosmetic preparation containing at least 5 wt. % of each thereof. The boiling point of the first solvent LM1, which is liquid at 20° C., at 1013.25 mbar is at a certain temperature x, whereas the boiling point of the second solvent LM2, which is liquid at 20° C., at 1013.25 mbar is then in the range from (x+5)° C. to (x+50)° C. or in the range from (x−50)° C. to (x−5)° C. Of course, the cosmetic preparations can also contain other solvents LM3, LM4, etc., but in this case it is not necessary to comply with the above-mentioned minimum amount of 5 wt. % or the boiling temperature differences.

Irrespective of the fact that it is possible to use further solvents LM3, etc., it is not preferable according to the invention to provide for their use in large amounts or even in amounts exceeding the amount of LM1 or LM2. It is preferred according to the invention if the cosmetic preparation a) contains at most 10 wt. %, preferably at most 7.5 wt. % and in particular at most 5 wt. % of solvents other than LM1 and LM2, which are liquid at 20° C.

The sprayability of the cosmetic preparations a) from the device b) is facilitated if the total amount of solvents LM1 and LM2 not only corresponds to the minimum amount of 10 wt. %, but LM1 and/or LM2 are used in larger amounts. Here, cosmetic products according to the invention are preferred in which the cosmetic preparation a) contains at least 10 wt. %, preferably at least 25 wt. %, more preferably at least 40 wt. % and in particular at least 50 wt. % of the first solvent LM1 which is liquid at 20° C.

Further preferred cosmetic products according to the invention are characterized in that the cosmetic preparation a) contains at least 10 wt. %, preferably at least 25 wt. %, more preferably at least 30 wt. % and in particular at least 35 wt. % of the second solvent LM2 which is liquid at 20° C.

It is particularly preferred to form as large a proportion as possible of the cosmetic preparation a) from LM1 and LM2. Here, cosmetic products according to the invention are preferred in which the cosmetic preparation a) contains—based on its weight—a total amount of solvent LM1 and solvent LM2 (LM1+LM2) of at least 40 wt. %, preferably of at least 50 wt. %, more preferably of at least 60 wt. %, even more preferably of at least 70 wt. % and in particular of at least 80 wt. %.

It has also proven preferable not to use as equal an amount as possible of the solvents, but to use a considerably larger amount of one of the solvents than of the other. Corresponding cosmetic products in which the weight ratio of the solvents to one another (the LM1/LM2 quotient) is >2, preferably >3, more preferably >4 and in particular >5, are preferred.

It has been found that the difference in the boiling points of solvents LM1 and LM2 at 1013.25 mbar in the range from 5 to 50° C. brings clear advantages when spraying the cosmetic preparation a) from the device b). The effects are particularly advantageous within a narrower temperature range. Here, cosmetic products according to the invention are preferred in which the difference between the boiling points of solvents LM1 and LM2 at 1013.25 mbar is in the range from 10 to 40° C., preferably in the range from 12.5 to 37.5° C., more preferably in the range from 15 to 35° C. and in particular in the range from 17.5 to 30° C.

In absolute terms, solvents with certain boiling points are particularly suitable for cosmetic applications. In terms of sprayability from the device b), it is particularly preferred if the boiling point of the first solvent LM1 at 1013.25 mbar is 70 to 90° C., preferably 72.5 to 87.5° C., more preferably 75 to 85° C. and in particular 77.5 to 82.5° C.

From the above-mentioned preferred boiling point differences, it thus also follows that it is particularly preferred if the boiling point of the second solvent LM2 at 1013.25 mbar is 90 to 110° C., preferably 92.5 to 107.5° C., more preferably 95 to 105° C. and in particular 97.5 to 102.5° C.

The cosmetic preparation a) sprayed by means of the device b) in the product according to the invention can be optimized and adapted to the device not only with regard to its physical properties, but also and especially with regard to its composition.

For certain fields of application such as hair masks, hair conditioners and other hair care compositions, emulsions have been shown to be particularly suitable as the cosmetic preparation in the product according to the invention. Oil-in-water emulsions (O/W emulsions) have proven particularly successful here, as their physical properties can be particularly effectively adapted to the device b).

Such emulsions preferably contain at least one emulsifier.

It has proven preferable within the context of the present invention for the cosmetic agent a) to contain a non-ionic emulsifier. According to the invention, non-ionic emulsifiers are understood to mean emulsifiers that have no charged groups. Charged groups are understood to be both permanently cationic and anionic groups and also temporarily cationic and anionic groups. Permanently cationic and anionic groups independent of the pH have a cationic or anionic charge. In contrast, temporarily cationic and anionic groups have a cationic or anionic charge only at certain pH values. Preferred cosmetic agents a) are therefore characterized in that they contain at least one emulsifier selected from the group of (i) addition products of 4 to 30 mol ethylene oxide and/or 1 to 5 mol propylene oxide and linear C₈-C₂₂ alcohols, C₁₂-C₂₂ carboxylic acids and C₈-C₁₅ alkylphenols, (ii) C₁₂-C₂₂ carboxylic acid mono- and diesters of addition products of 1 to 30 mol ethylene oxide and C₃-C₆ polyols, (iii) ethylene oxide and polyglycerol addition products with methyl glucoside carboxylic acid esters, carboxylic acid alkanolamides and carboxylic acid glucamides, C₈-C₂₂ alkyl mono- and oligoglycosides, (iv) addition products of 5 to 60 mol ethylene oxide and castor oil and hydrogenated castor oil, (v) partial esters of polyols having 3 to 6 carbon atoms with saturated C₈-C₂₂ carboxylic acids, (vi) sterols, (vii) carboxylic acid esters of sugars and sugar alcohols, and (viii) mixtures thereof.

The at least one emulsifier is advantageously used in certain quantity ranges in the cosmetic agents a). Preferred cosmetic agents a) are therefore characterized in that they contain—based on their total weight—0.1 to 40 wt. %, preferably 0.3 to 35 wt. %, preferably 0.5 to 30 wt. %, in particular 1.0 to 20 wt. %, of at least one emulsifier. The use of the quantities listed above ensures sufficient emulsification of the constituents and thus allows for high storage stability of the cosmetic agents.

Cosmetic agents a) in the form of an O/W emulsion contain at least one emulsified compound selected from the group of oils, waxes, esters or mixtures thereof.

It has proven advantageous within the scope of the present invention if the cosmetic agents a) contain at least one cosmetic oil, preferably at least one cosmetic non-silicone oil and/or one vegetable oil. According to the invention, “volatile non-silicon oils” are understood to mean oils which do not contain any silicon atoms and have, at 20° C. and an ambient pressure of 1,013 hPa, a vapor pressure of 2.66 Pa to 40,000 Pa (0.02 to 300 mm Hg), preferably from 10 to 12,000 Pa (0.1 to 90 mm Hg), more preferably from 13 to 3,000 Pa (0.1 to 23 mm Hg), in particular from 15 to 500 Pa (0.1 to 4 mm Hg). It is therefore preferred if the cosmetic agent a) contains at least one oil, wherein the oil is selected from the group of (i) volatile non-silicone oils, in particular liquid paraffin oils and isoparaffin oils, such as isodecane, isoundecane, isododecane, isotridecane, isotetradecane, isopentadecane, isohexadecane and isoeicosane; (ii) vegetable oils, in particular sunflower oil, olive oil, soybean oil, rapeseed oil, almond oil, jojoba oil, orange oil, wheat germ oil, peach kernel oil and the liquid portions of coconut oil; and (iii) mixtures thereof. The use of the aforementioned oils in the cosmetic agents a) leads to a high care effect and conditioning of the skin and/or hair.

Furthermore, it is preferred within the scope of the present invention if the cosmetic agents a) contain at least one wax. Preferred cosmetic agents a) are therefore characterized in that they contain at least one wax, wherein the wax is selected from the group of (i) coconut fatty acid glycerol mono-, di- and triesters; (ii) Butyrospermum Parkii (shea butter); (iii) esters of saturated, monohydric C₈₋₁₈ alcohols with saturated C₁₂₋₁₈ monocarboxylic acids; (iv) linear, primary C₁₂-C₂₄ alkanols; (v) esters of a saturated, monohydric C₁₆-C₆₀ alkanol and a saturated C₈-C₃₆ monocarboxylic acid, in particular cetyl behenate, stearyl behenate and C₂₀-C₄₀ alkyl stearate; (vi) glycerol triesters of saturated linear C₁₂-C₃₀ carboxylic acids, which may be hydroxylated, in particular hydrogenated palm oil, hydrogenated coconut oil, hydrogenated castor oil, glyceryl tribehenate and glyceryl tri-12-hydroxystearate; (vii) natural vegetable waxes, in particular candelilla wax, carnauba wax, Japan wax, sugar cane wax, ouricuri wax, cork wax, sunflower wax, fruit waxes; (viii) animal waxes, in particular beeswax, shellac wax and spermaceti; (ix) synthetic waxes, in particular montan ester waxes, hydrogenated jojoba waxes and sasol waxes, polyalkylene waxes and polyethylene glycol waxes, C₂₀-C₄₀ dialkyl esters of dimer acids, C₃₀₋₅₀ alkyl beeswax and alkyl and alkylaryl esters of dimer fatty acids, paraffin waxes; and (x) mixtures thereof. Particularly preferred are commercial products having the INCI name Cocoglycerides, especially the commercial products Novata® (ex BASF), particularly preferred Novata® AB, which is a mixture of C₁₂₋₁₈ mono-, di- and triglycerides and has a melting point range of 30 to 32° C., as well as the products of the Softisan range (Sasol Germany GmbH) having the INCI name Hydrogenated Coco-glycerides, in particular Softisan 100, 133, 134, 138, 142. Other preferred esters of saturated, monohydric C₁₂₋₁₈ alcohols with saturated C₁₂₋₁₈ monocarboxylic acids are stearyl laurate, cetearyl stearate (e.g., Crodamol® CSS), cetyl palmitate (e.g., Cutina® CP) and myristyl myristate (e.g., Cetiol® MM). Furthermore, a C₂₀-C₄₀ alkyl stearate is preferably used as a wax component. This ester is known under the name Kester Wax® K82H or Kester Wax® K80H and is distributed by Koster Keunen Inc.

In addition, it has proven advantageous within the scope of the present invention if the cosmetic agent a) contains at least one ester. It is therefore preferred according to the invention if the cosmetic agent contains at least one ester, wherein the ester is selected from the group of (i) triethyl citrates, (ii) dicarboxylic acid esters of linear or branched C₂-C₁₀ alkanols, (iii) symmetrical, asymmetrical or cyclic esters of carbonic acid with alcohols, (iv) esters of dimers of unsaturated C₁₂₋₂₂ carboxylic acids with monovalent, linear, branched and cyclic C₂₋₁₈ alkanols or C₂₋₆ alkanols, (v) benzoic acid esters of linear or branched C₈₋₂₂ alkanols, such as benzoic acid C₁₂₋₁₅ alkyl esters and benzoic acid isostearyl esters and benzoic acid octyldodecyl esters; and (vi) mixtures thereof. The use of the previously mentioned esters also leads to effective care and conditioning of the skin and/or hair.

Particularly preferred embodiments of the present invention contain at least one of an oil and/or wax and/or ester mentioned above.

In summary, cosmetic products according to the invention are preferred in which the cosmetic preparation a) is an oil-in-water (O/W) emulsion which contains, based on its weight, 1 to 25 wt. % of cosmetic oil(s).

In addition to the components already described above, the cosmetic agents a) can in principle be used all other components known to a person skilled in the art for such cosmetic compositions. Other active substances, auxiliary substances, and additives are, for example:
thickening agents, such as gelatin or vegetable gum, for example agar-agar, guar gum, alginates, xanthan gum, gum arabic, karaya gum, locust bean flour, linseed gum, dextrans, cellulose derivatives, for example methyl cellulose, hydroxyalkyl cellulose and carboxymethyl cellulose, starch fractions and derivatives, such as amylose, amylopectin and dextrins, fully synthetic hydrocolloids, such as polyvinyl alcohol,
structurants such as maleic acid and lactic acid,
solvents and solubilizers, such as ethanol, isopropanol, ethylene glycol, propylene glycol, glycerol, and diethylene glycol,
fiber structure-improving active constituents, in particular mono-, di-, and oligosaccharides, such as glucose, galactose, fructose, fruit sugar, and lactose,
dyes for coloring the agent,
substances for adjusting the pH, such as α and β hydroxycarboxylic acids,
active substances such as allantoin and bisabolol,
complexing agents such as EDTA, NTA, β-alanine diacetic acid, and phosphonic acids,
ceramides. Ceramides are understood to mean N-acylsphingosine (fatty acid amides of sphingosine) or synthetic analogs of such lipids (so-called pseudo-ceramides),
opacifying agents, such as latex, styrene/PVP and styrene/acrylamide copolymers,
pearlescent agents, such as ethylene glycol mono- and distearate and PEG-3 distearate,
pigments,
viscosity regulators such as salts (NaCl),
anionic, cationic and amphoteric surfactants,
cationic, nonionic, and amphoteric polymers,
vitamins, in particular from groups A, B, C, E, F, and H,
UV filters, in particular benzophenone, p-aminobenzoic acid esters, diphenylacrylic acid esters, cinnamic acid esters, salicylic acid esters, benzimidazoles, and o-aminobenzoic acid esters,
protein hydrolysates and cationized protein hydrolysates,
humectants or penetration aids and/or bulking agents, in particular urea and urea derivatives, guanidine and derivatives thereof, arginine and derivatives thereof, water glass, imidazole and derivatives thereof, histidine and derivatives thereof, benzyl alcohol, glycol ethers, propylene glycol ethers, for example propylene glycol monoethyl ether, carbonates, hydrogen carbonates, 1,2-diols, and 1,3-diols,
plant extracts, for example from green tea, white tea, oak bark, stinging nettle, witch hazel, hops, chamomile, burdock root, horsetail, whitethorn, lime blossom, lychee, almond, aloe vera, spruce needle, horse chestnut, sandalwood, juniper, coconut, mango, apricot, lemon, wheat, kiwi, melon, orange, grapefruit, sage, rosemary, birch, mallow, cuckoo flower, wild thyme, yarrow, thyme, melissa, restharrow, coltsfoot, marshmallow, ginseng, ginger, Echinacea purpurea, Olea europaea, Foeniculum vulgaris and Apium graveolens,
silicone oils, in particular polyalkylsiloxanes, polyarylsiloxanes, and polyalkylarylsiloxanes, which can optionally be functionalized with organic groups and/or ethoxy and/or propoxy groups.

The aforementioned other constituents can be contained, based on the total weight of the cosmetic agent, in a total amount of 0.001 to 50 wt. %, preferably of 0.01 to 40 wt. %, preferably of 0.1 to 30 wt. %, in particular of 0.5 to 20 wt. %.

The cosmetic product according to the invention comprises a device for the finely distributed application of the cosmetic preparation a). This device in turn comprises
b1) a storage container for the cosmetic preparation a),
b2) pump or a conveying element operating in a similar manner for conveying the cosmetic preparation a) out of the storage container b1);
b3) a pressure chamber in which the cosmetic preparation a) conveyed out of the storage container b1) can be subjected to a pressure increase, comprising
   b3a) an inlet valve or a closure element operating in a similar manner in order to be able to seal the cosmetic preparation a) conveyed by means of the conveying element b2) from the storage container b1) into the pressure chamber b3) with respect to the environment;
   b3b) an outlet valve or a closure element operating in a similar manner in order to be able to apply the cosmetic preparation a) from the pressure chamber b3) that is under increased pressure in a finely distributed manner,

The storage container b1) serves as a storage receptacle for the cosmetic composition a). It may be rigidly connected to the device, but a detachable and replaceable design is preferred, which makes it possible to connect different storage containers to one device and thus also to spray different cosmetic preparations. The volume of the storage container b1) is ideally adapted to the intended use and depends on the amount of cosmetic preparation to be applied and on the desired number of applications that a storage container should provide once filled.

Standard volumes for storage containers are in the range of 1 to 500 ml, preferably 2 to 250 ml and in particular 5 to 500 ml.

The device of the product according to the invention further comprises a pump b2) or a conveying element operating in a similar manner for conveying the cosmetic preparation a) out of the storage container b1). The conveying element b2) can be located directly in the storage container b1) or connected thereto via suitable lines, for example riser pipes. There are no limits to the technical and visual design of the (preferably replaceable) storage container b1) and conveying element b2).

If a pump is used as the conveying element, both dynamic and positive displacement pumps can be used. In flow pumps such as axial, diagonal and radial pumps, energy transfer is achieved through fluidic processes, while in positive displacement pumps the medium is conveyed through self-contained volumes. Diaphragm pumps, rotary piston pumps such as lobe pumps, rotary vane pumps, circumferential piston pumps and rotary gear pumps, eccentric screw pumps, impeller pumps, chain pumps, piston pumps, peristaltic pumps and screw pumps are particularly suitable here.

The device of the product according to the invention further comprises a pressure chamber b3) in which the cosmetic preparation a) conveyed out of the storage container b1) can be subjected to an increase in pressure. In order for the cosmetic preparation a) to be repeatedly conveyed out of the storage container b1) by the conveying element b2) and subsequently sprayed, the pressure chamber b3) comprises an inlet valve b3a) or a closure element operating in a similar manner in order to be able to seal the cosmetic preparation a) conveyed by means of the conveying element b2) from the storage container b1) into the pressure chamber b3) with respect to the environment and an outlet valve b3b) or a closure element operating in a similar manner in order to be able to apply the cosmetic preparation a) from the pressure chamber b3), which is under increased pressure, so as to be finely distributed.

The closure elements b3a) and b3b) are preferably controllable valves in which a closure part (for example a plate, a cone, a ball or a needle) is moved approximately in parallel with the flow direction of the fluid. The flow is interrupted by pressing the sealing surface of the closure part against a matching opening, the so-called sealing seat. With regard to the desired automatic application of the cosmetic preparation a), electric motor-driven or electromagnetically operated valves with short closing and opening times are preferred.

According to the present invention, a device for spraying a cosmetic preparation is provided which allows the temperature and/or pressure of the cosmetic product to be increased when the inlet valve b3a) and the outlet valve b3b) are closed, causing at least some of the cosmetic preparation a) in the chamber to change state.

The cosmetic composition a) conveyed by the conveying element b2) into the pressure chamber b3) may be a liquid or a mixture of liquid and gas, such as a foam, with liquids being preferred. The cosmetic preparation a) may also contain distributed, particulate solids.

The pressure chamber b3) comprises an inlet valve b3a) and an outlet valve b3b), which are preferably provided at separate, different locations in the pressure chamber. When the temperature and pressure in the chamber increase, a liquid cosmetic composition a) in the chamber changes its state partially to a gas which is partially distributed in the liquid, also forming a foam. Preferably, the inlet valve b3a) and the outlet valve b3b) each comprise an actuator and a seat. The actuating drive can control the opening and closing of the valve. The actuator can be a solenoid. The valve seat may provide a sealing surface allowing the valve to close and the chamber to be pressurized. The cosmetic composition a) is fed from the storage container b1) to the pressure chamber b3), where it is pressurized. The pressure increase can be achieved by any means known to a person skilled in the art, for example by pressure being applied using a compressed gas, by reducing the volume of the pressure chamber, by means of shock waves, or by heating, said heating being preferred on account of being simple to be implement in terms of apparatus.

The pressure chamber b3) is made of a material that can withstand considerable temperature changes and pressure differences. It can have a generally cylindrical shape. The pressure chamber b3) can be made of a metal such as steel, copper or aluminum or a polymer. Alternatively, the pressure chamber b3) may be formed from a composite material wrapped around a metal lining in the form of a wrapped pressure container made of composite material. The pressure chamber b3) can be lined with a different metal, ceramic or polymer. The size and shape of the pressure chamber b3) can vary depending on the desired application.

The pressure chamber b3) may further comprise equipment for conducting and controlling the fluid flow of cosmetic composition a) from the inlet valve b3a) to the outlet valve b3b). Such devices are arranged inside the chamber and direct the cosmetic composition so that it preferably follows a non-linear path. The means for directing and controlling the fluid flow of cosmetic composition a) from the inlet valve b3a) to the outlet valve b3b) along a preferably non-linear path can initiate and assist the formation of foam inside the chamber and help to ensure that foam is present inside the chamber at the outlet valve b3b), resulting in improved sprayability and a more homogeneous spray pattern.

The inclusion of a means for directing and controlling the fluid flow of cosmetic composition a) from the inlet valve b3a) to the outlet valve b3b) along a preferably non-linear path inside the chamber causes an obstruction inside the chamber, thereby preventing the liquid from moving freely through the chamber when the pressure chamber b3) or the device b) is moved. With regard to cosmetic applications where the position of the device b) and the discharging direction of the outlet valve b3b) change continuously during the application (e.g., when applying a hairspray to the user's head), an obstacle slows down the fluid of cosmetic composition a) from the inlet valve b3a) to the outlet valve b3b) so as to reduce the stimulus or impact on the foam, and the foam is largely protected from destruction regardless of the movement of the pressure chamber b3).

The direction of the fluid consisting of cosmetic composition a) from the inlet valve b3a) to the outlet valve b3b) along a preferably non-linear path thus ensures that a pool of liquid does not accumulate in front of the outlet valve b3b), therefore allowing efficient and effective operation of the device b). It is desirable according to the invention to have a high foam concentration near the outlet valve b3b) of the pressure chamber b3) since it has been found that when foam rather than liquid is discharged from the outlet valve b3b), the size of the resulting spray droplets is smaller. Further breakup of the foam occurs when the fluid is discharged through the outlet valve b3b) by a vapor explosion.

Due to these device measures, the properties of the spray discharged from the outlet valve b3b) remain largely unaffected, even if the device b) is moved or its orientation is changed. This improves the reproducibility of the spray characteristics achieved with a given set of conditions, which in turn also improves the reliability of the device b) in any given application.

Before pressurizing the cosmetic composition a) in the pressure chamber b3) (preferably before heating), the inlet b3a) and outlet b3b) valves are closed to prevent of cosmetic preparation a) from escaping. The increase in pressure (preferably heating) of the cosmetic composition a) in the pressure chamber b3) causes an increase in the pressure inside the chamber and thus also a reduction in the boiling point of the cosmetic composition a). In most cases where the cosmetic composition a) in the pressure chamber b3) becomes a foam when pressurized (preferably by heating), the saturation or boiling point of the fluid consisting of cosmetic composition a) is based on the boiling temperature of the liquid phase. The cosmetic composition a) is heated to a temperature well above the boiling point at atmospheric pressure, causing the cosmetic composition a) to change state.

The temperature inside the pressure chamber b3) can be monitored by one or more temperature sensors. A means for heating the cosmetic preparation a) may be a heating element located in or near the chamber for heating the cosmetic agent a). The heating means can, for example, be a heated jacket which surrounds or partially surrounds the pressure chamber b3). Alternatively, the heating medium can be generated by chemical components. For example, two chemicals can be combined which, when mixed, undergo an exothermic reaction, whereby the heat generated is sufficient to heat the cosmetic preparation a) to a temperature which exceeds the saturation temperature of the liquid.

The sudden pressure relief when the cosmetic preparation a) exits the outlet valve b3b) causes a vapor explosion due to the rapid expansion of liquid, foam and/or vapor. The vapor explosion causes the material to be transported out of the pressure chamber b3) very quickly and over greater distances than would otherwise be possible. A mixture of vapor and fine spray mist is discharged from the outlet valve b3b), which mixture can spread out at high speeds and over considerable distances. For example, the throwing distance of a liquid and vapor explosion according to embodiments of the present invention may be about 200 to 300 times the corresponding length of the pressure chamber, or more. This is due to the high fluid pressures obtained in the pressure chamber b3) as well as the dynamics of the fluid inside the chamber.

One advantage of the device b) consists in that it can deliver continuous bursts of vapor in very rapid succession. The control of the inlet valve b3a) and outlet valve b3b) can be programmed so that the outlet valve b3b) opens every few milliseconds. The temperature at which the outlet valve b3b) may open can be referred to as the trigger temperature. The trigger temperature can be set to be above the boiling point of the liquid or liquids in the chamber to ensure maximum explosion of the liquid from the chamber. The trigger temperature can be set to be in the range of 10° C. to 200° C. above the boiling point of the liquid. Preferably, the trigger temperature is set to be in the range of 20° C. to 90° C. above the boiling point of the liquid. The trigger temperature required is relative to the ambient pressure of the environment into which the discharged spray is sprayed, i.e., the region outside the chamber at the outlet opening. When the ambient pressure is high, the temperature and pressure in the chamber must be increased and the trigger temperature value is at the upper end of the range. The liquid to vapor ratio can be changed if higher trigger temperatures are selected. This can completely eliminate the liquid phase, if desired. In this way, the liquid to vapor ratio can be controlled by varying one or more parameters associated with the chamber. It has been found that the droplet size is large if the trigger temperature is not at least 10° C. higher than the boiling point of the liquid.

Alternatively, instead of monitoring the temperature, the pressure inside the chamber can be monitored and the outlet valve b3b) can be opened when a predetermined pressure value is reached. Selectively varying one or more parameters such as temperature, pressure or viscosity of the cosmetic composition a) can be used to selectively control the droplet size achieved in the resulting spray. The size of the outlet opening can vary depending on the desired spray characteristics. The outlet opening out of the pressure chamber b3) may be connected to a nozzle to change the dispersion properties of the spray. The nozzle can be used to produce a spray mist having a wider scattering range or a narrower, more concentrated spray mist. A nozzle can also be used to further reduce the droplet size of the liquid in the spray, thereby producing a finer spray.

The preferably non-linear path along which the cosmetic preparation a) is guided inside the pressure chamber b3) can cause a change in the direction in which the cosmetic preparation b) is moving by at least 90°. The required degrees to which said direction is changed depend on the application or end use of the device b). The non-linear path could cause a change in the direction in which the cosmetic preparation a) is moving by at least 180°, 270° or 360°. Depending on the application, it may be necessary to increase the degrees to which the direction is changed in order to protect the foam layer inside the chamber from the movement of the fluid.

For applications where the device b) may be subjected to greater degrees of movement, it is preferable to conduct the cosmetic preparation a) along a more complex or more tortuous path to provide a change of at least 180°. The goal of a non-linear path is to prevent liquid from moving rapidly in a wave motion inside the pressure chamber b3). The more the liquid flow is interrupted, the less kinetic energy the liquid has when it comes into contact with the foam, which in turn results in more of the foam being retained. For example, if the pressure chamber b3) is subjected to a rocking motion along a single axis, it would be sufficient for the non-linear path to direct the fluid through a change of at least 90°.

For example, a baffle or barrier inside the chamber could change the direction of movement of the fluid by 90° so as to bypass the baffle. If in practice the pressure chamber b3) is only subjected to a rocking motion, the deflection plate can be arranged such that liquid is retained on one side of the barrier with only foam or gas easily flowing over the deflection plate. This depends on the relative height and arrangement of such a baffle plate. The baffle would have to cause a change in the direction of the fluid of at least 90° to achieve the desired effect. Such an arrangement of the baffle plate can prevent the cosmetic preparation a) on the first side of the baffle plate from destroying or breaking up any foam that may be present on the second side of the baffle plate, despite the movement of the chamber. For applications where the chamber is subjected to larger degrees of movement, possibly along more than one axis, larger degrees of change in the direction of the non-linear path will be necessary to prevent the foam from being destroyed. Some applications require a change of at least 180°, and others require a change of at least 360°.

A preferably non-linear path inside the pressure chamber b3) may comprise at least one non-linear channel and may comprise a plurality of non-linear channels. In general, a single channel is preferable when the cosmetic preparation is a) viscous. The means for directing and controlling the flow of fluid from the inlet opening to the outlet opening may comprise at least one channel having a series of bends that cause the fluid to change direction a number of times. The fluid can be conducted along a winding path that has many bends at different angles. The means for directing and controlling the fluid flow of cosmetic composition a) from the inlet valve b3a) to the outlet valve b3b) may comprise at least one spiral-shaped or helical channel. As a result of the channel, the fluid can be guided along an oscillating or winding path. The means for conducting and controlling the flow of fluid consisting of cosmetic composition a) from the inlet valve b3a) to the outlet valve b3b) along a non-linear path could comprise at least one baffle arranged to cause the fluid to change direction. Optionally, it could comprise a series of deflectors arranged to cause the fluid to change direction multiple times. The baffles are preferably arranged inside the pressure chamber b3) to prevent the fluid from following a linear path between the inlet valve b3a) and the outlet valve b3b).

As already mentioned, the pressure increase in the pressure chamber b3) is preferably achieved by heating. At least one means for heating the cosmetic preparation a) may be located outside the pressure chamber b3). The heating means may, for example, be a heated jacket that surrounds or partially surrounds the chamber. This could be used in isolation or in conjunction with other heating means, such as a heating means located inside the chamber. At least one means for heating the fluid may be located inside the pressure chamber b3).

For example, a helical channel can be formed around a central cylindrical heating element. As a further option, at least one means for heating the fluid may be located inside the chamber and the means for directing and controlling the fluid flow from the inlet opening to the outlet opening along a non-linear path may be positioned inside the heating means where the fluid flow is fluidically isolated from the heating means. For example, a heating coil may be configured to abut the inner walls of the chamber, and this heating coil may be filled with a shaped element that ensures that the fluid flow is conducted along a non-linear path from the inlet valve b3a) to the outlet valve b3b).

The device b) preferably comprises at least one controller connected to the inlet valve b3a) and the outlet valve b3b), so that opening and closing of the inlet valve b3a) and the outlet valve b3b) is controlled electronically. The controller can be programmed to close the outlet valve b3b) when the closing pressure or a set temperature is reached and to reopen the inlet valve b3a) to introduce new fluid into the chamber. The system can alternate between introducing new fluid into the chamber and discharging the fluid out of the outlet opening. Alternatively, the valve controller can be offset such that the pressure chamber b3) is filled with fluid and the outlet valve b3b) then makes a series of short, rapid opening operations until the pressure chamber b3) is empty. The controller may be programmed to open and close the valves b3a) and b3b) according to a time sequence, wherein the valves are opened and closed for a predetermined amount of time provided that a predetermined (or set) pressure or predetermined temperature within the pressure chamber b3) has been reached or exceeded.

The predetermined temperature could correspond to the saturation temperature of the fluid inside the chamber at atmospheric pressure. The temperature may be monitored by one or more temperature sensors, which may be attached inside the chamber or near the chamber, for example in the inlet stream, or on a wall of the chamber. The device may also comprise at least one pressure sensor inside the chamber. This can be a pressure transducer. When fluid is discharged from the pressure chamber b3), the pressure inside the chamber drops. The outlet valve b3b) may be arranged so as to close when the pressure has fallen back to an ambient or second predetermined pressure, which may be referred to as the closing pressure. Alternatively, the outlet valve b3b) may be arranged so as to close after a preselected period of time has elapsed.

It is possible to provide a recirculation loop from the pressure chamber b3) to the storage container b1). The recirculation loop would be designed to allow some of the fluid from the pressure chamber b3) to return to the storage container b1) when the inlet valve b3a) is open to top up the cosmetic agent a) in the pressure chamber b3). The recirculation line allows some fluid to flow from the pressure chamber b3) back to the storage container b1). Fresh cosmetic preparation a) is fed from the storage container b1) to the pressure chamber b3) via the inlet valve b3a) by means of the conveying element b2). The returned cosmetic preparation a) is warmer than the cosmetic preparation a) in the storage container b1), and therefore the recirculation helps to increase the temperature of the cosmetic preparation a) in the storage container b1). This in turn can accelerate heating of the cosmetic preparation a) in the pressure chamber b3).

A method is also provided for spraying a cosmetic preparation, in which a cosmetic preparation a) is conveyed out by means of a device for the finely distributed application of the cosmetic preparation a) from a storage container b1) by means of a pump or a conveying element operating in a similar manner in order to convey the cosmetic preparation a) out of the storage container b1); and is transferred into a pressure chamber in which the cosmetic preparation a) conveyed out of the storage container b1) a) can be subjected to an increase in pressure, wherein the pressure chamber b3) comprises an inlet valve or a closure element operating in a similar manner in order to be able to seal the cosmetic preparation a) conveyed by means of the conveying element b2) from the storage container b1) into the pressure chamber b3) a) with respect to the environment; and furthermore an outlet valve or a closure element operating in a similar manner in order to be able to apply the cosmetic preparation a) from the pressure chamber b3), which is under increased pressure, so as to be finely distributed, wherein the cosmetic preparation a) has a degree of thermal conductivity of 0.02 to 2.0 Wm⁻¹K⁻¹ at 25° C.

The above description of the device also applies, mutatis mutandis, to the spraying method.

1. A cosmetic product, comprising:
a cosmetic preparation; and
a device configured to apply the cosmetic preparation in a finely distributed manner, the device comprising:
a container storing the cosmetic preparation;
a conveying element configured to convey the cosmetic preparation out of the container; and
a pressure chamber configured to receive the conveyed cosmetic preparation and to subject the conveyed cosmetic preparation to a pressure increase, the pressure chamber comprising:
an inlet valve configured to seal the conveyed cosmetic preparation within the pressure chamber with respect to the environment; and
an outlet valve configured to apply the cosmetic preparation that is under increased pressure in a finely distributed manner,
wherein the cosmetic preparation has a degree of thermal conductivity of from 0.02 to 2.0 Wm⁻¹K⁻¹ at 25° C.

2. A method for applying a cosmetic preparation in a finely distributed manner using the device of the cosmetic product of claim 1.

3. The cosmetic product of claim 1, wherein the cosmetic preparation has a degree of thermal conductivity of from 0.05 to 1.5 Wm⁻¹K⁻¹ at 25° C.

4. The cosmetic product of claim 1, wherein the cosmetic preparation has a specific heat capacity of from 1.6 to 5.2 Jg⁻¹K⁻¹ at 20° C.

5. The cosmetic product of claim 1, wherein the cosmetic preparation has a volumetric coefficient of thermal expansion g (20° C.) of from 0.0001 to 0.005 K⁻¹.

6. The cosmetic product of claim 1, wherein the cosmetic preparation has a vapor pressure of from 1200 to 8000 Pa at 20° C. according to the Grain-Watson model.

7. The cosmetic product of claim 1, wherein the cosmetic preparation has a viscosity of from 500 to 25000 mPas at 20° C. according to Brookfield DV2T, 20 rpm, spindle 5.

8. The cosmetic product of claim 1, wherein the cosmetic preparation has a viscosity of from 10 to 100 mPas at 20° C. according to Brookfield DV2T, 10 rpm, spindle 2.

9. The cosmetic product of claim 1, wherein the cosmetic preparation is an oil-in-water emulsion comprising 1 to 25 wt. % of at least one cosmetic oil based on the total weight of the cosmetic preparation.

10. The cosmetic product of claim 1, wherein the cosmetic preparation comprises at least 5 wt. % of a first solvent which is liquid at 20° C. and at least 5 wt. % of a second solvent which is liquid at 20° C., based on the total weight of the cosmetic preparation.

11. The cosmetic product of claim 1, wherein the conveying element is a pump.

12. The cosmetic product of claim 3, wherein the cosmetic preparation has a degree of thermal conductivity of from 0.1 to 1.0 Wm⁻¹K⁻¹ at 25° C.

13. The cosmetic product of claim 12, wherein the cosmetic preparation has a degree of thermal conductivity of from 0.2 to 0.6 Wm⁻¹K⁻¹ at 25° C.

14. The cosmetic product of claim 4, wherein the cosmetic preparation has a specific heat capacity of from 3.1 to 4.3 Jg⁻¹K⁻¹ at 20° C.

15. The cosmetic product of claim 5, wherein the cosmetic preparation has a volumetric coefficient of thermal expansion g (20° C.) of from 0.0002 to 0.0011 K⁻¹.

16. The cosmetic product of claim 6, wherein the cosmetic preparation has a vapor pressure of from 2200 to 5600 Pa at 20° C. according to the Grain-Watson model.

17. The cosmetic product of claim 7, wherein the cosmetic preparation has a viscosity of from 2000 to 10000 mPas at 20° C. according to Brookfield DV2T, 20 rpm, spindle 5.

18. The cosmetic product of claim 10, wherein the difference between the boiling point of the first solvent and the boiling point of the second solvent at 1013.25 mbar is from 5 to 50° C.

19. The cosmetic product of claim 10, wherein the cosmetic preparation further comprises at most 10 wt. % of a third solvent which is liquid at 20° C., based on the total weight of the cosmetic preparation.

20. The cosmetic product of claim 1, wherein the cosmetic preparation comprises, based on the total weight of the cosmetic preparation:
at least 50 wt. % of a first solvent which is liquid at 20° C.;
at least 35 wt. % of a second solvent which is liquid at 20° C.; and
at most 5 wt. % of a third solvent which is liquid at 20° C.
